(19) United States
(12) Patent Application Publication
(10) Pub. No.: US 2026/0224873 A1
(43) Pub. Date: Aug. 6, 2026

(54) Hydro Dermabrasion Device With Microneedles

(71) Applicant: Lawrence G Groop, Two Harbors, MN (US)

(72) Inventor: Lawrence G Groop, Two Harbors, MN (US)

(21) Appl. No.: 19/351,474

(22) Filed: Oct. 7, 2025

Related U.S. Application Data
(63) Continuation-in-part of application No. 19/026,053, filed on Jan. 16, 2025, now Pat. No. 12,458,403.

(51) Int. Cl. A61M 37/00; A61B 17/00

(52) U.S. Cl. CPC A61M 37/0015; A61B 17/00; A61B 2017/00761; A61B 2217/005; A61M 2037/0061

(57) ABSTRACT

An applicator tip for a hydro dermabrasion device includes a retractable microneedle array in which the penetration depth of the microneedles may be variably controlled.

### TECHNICAL FIELD OF THE INVENTION

The disclosed device relates to the application of solutions from a device with a motorized pump, handpiece, and specialized tip with the intent of exfoliating the skin while simultaneously incorporating an array of microneedles in this tip to assist in the exfoliation. Further embodiments include components that enhance the comfort and efficiency of the exfoliation.

### BACKGROUND OF THE INVENTION

Currently, clinicians use “hydro dermabrasion” as a method of exfoliating and beautifying the skin. The apparatus includes at least two motorized pump units with fluid solution reservoirs, a hose that connects the supply and suction pumps to a handpiece, and a single use “tip” attachment that has tubes through it (connected to the supply pump tube and reservoir). One tube allows fluid to enter the tip and express onto the skin on one side of the tip via an aperture. On the other side of tip is another tube and aperture that allows fluids and skin cell remnants to be suctioned away from the surface of the skin into the suction tube that is connected to the suction pump that stores the spent fluid into a reservoir inside the machine.

Another method of exfoliating the skin is to use metal microneedles arranged on an array and then moving the array around the skin which allows the needles to exfoliate the top layer of the epidermis - stratum corneum. It is necessary to use a “glide serum” or whetting agent to reduce the friction that the microneedle array has when held perpendicular and moved across the surface of the skin.

### SUMMARY OF THE DISCLOSURE

This invention combines both exfoliation technologies into one single-use “tip” attachment to the handpiece. It leverages the liquid exfoliant solution to also act as a glide serum for the motion of the perpendicular tip across the surface of the skin.

An applicator assembly attaches to a hydro dermabrasion handpiece, which may be of different designs and configurations, with two lumens that align with supply and suction lumens on the handpiece interface. The lumens pass through the applicator tip to two apertures (supply and suction) on the surface of a protective plate through which, when the clinician pushes down on the handpiece, microneedles emerge through guide holes in a protective plate and assist in the exfoliation of the skin. This sequence can occur by itself or simultaneously with the expression and suction of the exfoliation solution of the hydro dermabrasion device.

In one embodiment, an applicator tip attaches to the handpiece interface by either threaded or bayonet insertion design. The applicator assembly allows fluid to be expressed onto the skin via the supply lumen and subsequently suctioned away from the skin through the return suction lumen, independent of the advancement or retraction of the microneedle array through the protective plate. A spring mechanism prevents the microneedle array from advancing through the protective plate without the downward, intentional pressure of the applicator tip against the skin. When the procedure is finished, the applicator tip is detached from the hydro dermabrasion handpiece and discarded.

### BRIEF DESCRIPTION OF DRAWINGS

FIG. 1 shows different embodiments of handpieces for use with hydro dermabrasion devices.

FIG. 2 shows a version of a handpiece for use with the current invention.

FIG. 3 shows a proximal end view of the handpiece of FIG. 2.

FIG. 4 shows a distal end view of the handpiece of FIG. 2.

FIG. 5 shows a handpiece with an embodiment of the current invention attached.

FIG. 6 shows an embodiment of an external vibration unit that may be attached to a handpiece for use with the current invention.

FIG. 7 shows a cut away view of an embodiment of the current invention.

FIG. 8 shows an exploded cut away view of the embodiment of FIG. 7.

FIG. 9 shows an end view of the embodiment of FIG. 7.

FIG. 10 shows an embodiment of the current invention with adjustable depth control.

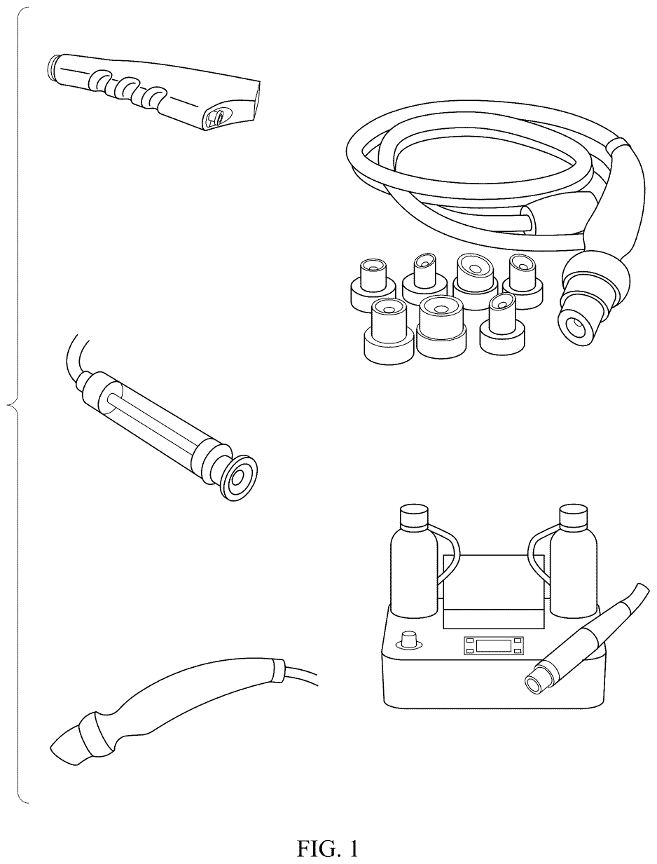

### DETAILED DESCRIPTION

To provide a more complete understanding of the present invention and features and advantages thereof, reference is made to the following description, taken in conjunction with the accompanying figures, wherein like reference numerals represent like parts.

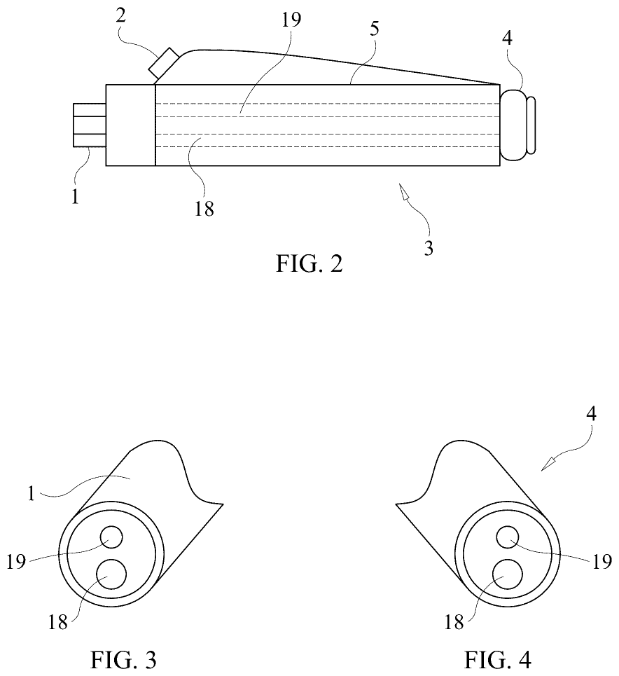

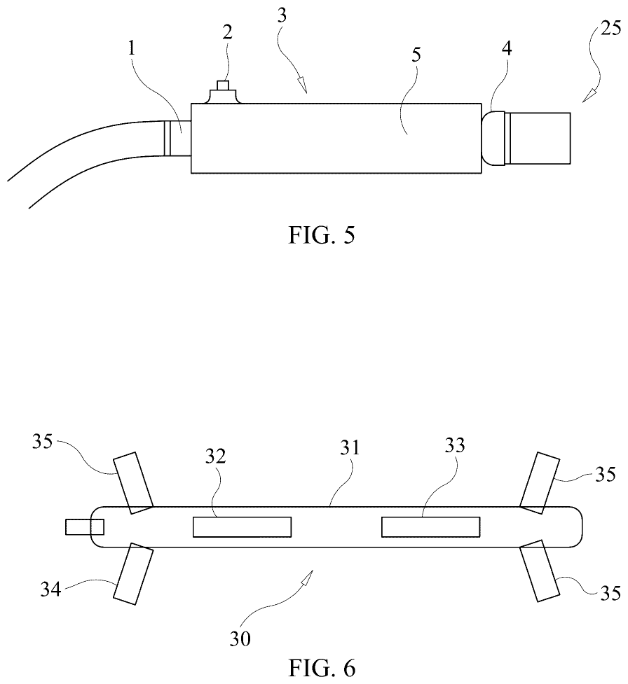

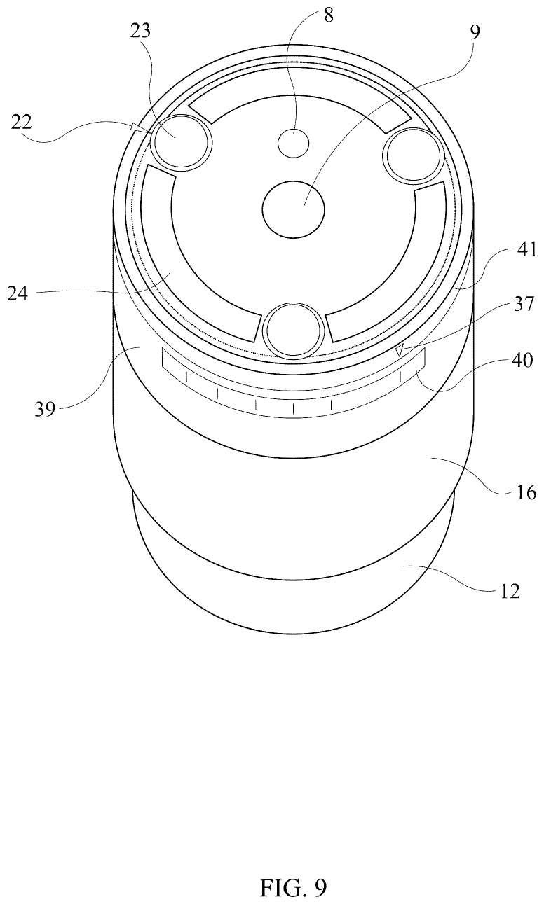

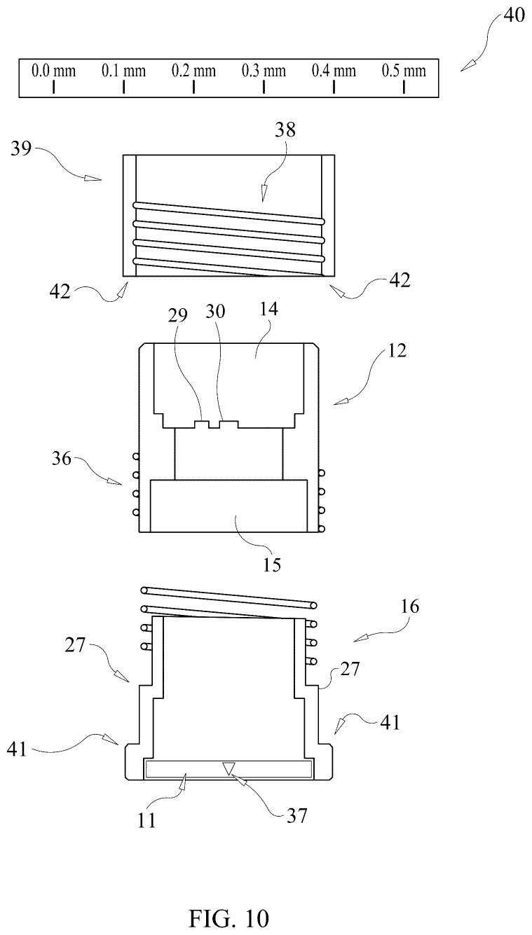

As seen in FIGS. 2-5, a hydro dermabrasion handpiece 3, to which the current invention attaches, comprises an elongated body 5 with a suction/supply hose interface 1 on its proximal end and a handpiece applicator tip interface 4 on its distal end. Interface 1 incorporates openings at the end of lumens that connect to a solution supply 19 and a suction tube 18 from the main hydro dermabrasion machine (not shown). Tubes 18 and 19 pass through the body 5 and emerge at the applicator tip interface 4, where they align with tubes or apertures in the applicator tip. In an embodiment of the current invention, tubes 18 and 19 align with suction lumen 6 and solution supply lumen 13, respectively, of applicator tip assembly 25 (FIGS. 5 & 7) and with distal openings 8 and 9 (FIG. 9). In the illustrated embodiment, the solution supply line 18,13,9 is near the central axis of the tip 25 and the suction line 19,6,8 is off to the side. This configuration helps more evenly distribute the solution along the skin. Other configurations, which may include more than one suction or supply line through the tip, may be deployed.

A dual pump actuation trigger 2, which turns on the vacuum pump and the liquid supply pump of the hydro dermabrasion machine, is preferably integral with the handpiece, although a switch on the machine may perform this function.

When using a hydro dermabrasion handpiece 3 with an embodiment of the current invention, it may be advantageous to apply vibrations to the handpiece. FIG. 6 shows a schematic embodiment of an external vibration unit 30 adaptable to the handpiece, comprising a main body cylinder 31 with a power supply (such as a battery) 32 and a vibration motor 33 and an on/off switch 34. Attachment tabs 35 may permanently or removably affix the vibrator to the handpiece.

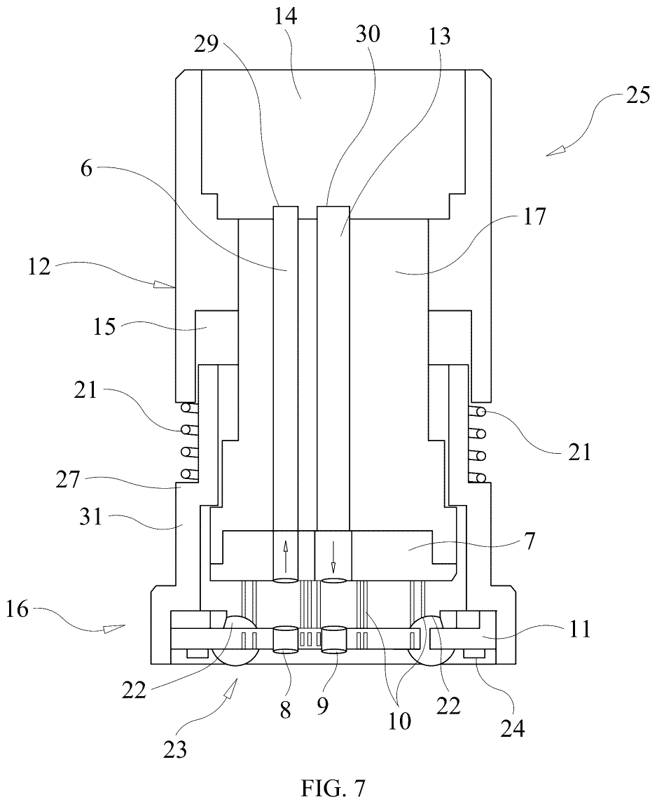

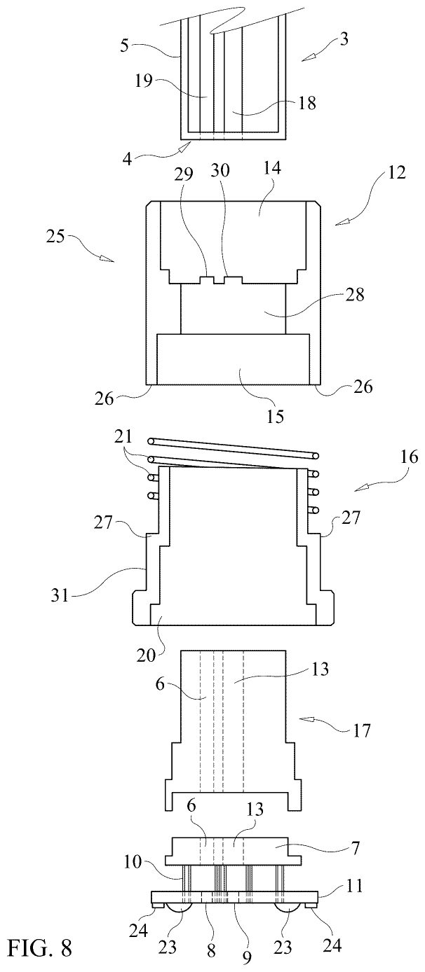

As shown in FIGS. 7-9, single-use applicator tip assembly 25 includes an upper cylinder body 12, a lower cylinder body 16 and a needle array base holder 17. The upper cylinder body 12 has an upper chamber 14 dimensioned to releasably attach to the handpiece 3. When the handpiece 3 is in position, the tip interface 4 aligns a solution supply tube 18 and a suction tube 19 with corresponding solution supply 30 and suction 29 interfaces in the upper cylinder body 12. In the illustrated embodiment, supply interface 30 and suction interface 29 extend upward and are inserted into the supply 19 and suction 19 tubes in tip interface 4. Other connections are possible to align the respective tubes. For example, tip interface 4 could have extensions of its tubes that fit into the tubes in upper cylinder body 12.

Upper cylinder body 12 also has a lower chamber 15 dimensioned to movably engage lower cylinder body 16. A spring 21 is positioned between the lower wall 26 of upper cylinder body 12 and a first external ledge 27 around the lower cylinder body 16 so that downward movement of the upper cylinder body relative to the lower cylinder body compresses the spring. The spring may be fixedly attached, with glue or another method, to the lower wall 26 and the ledge 27 in order to prevent separation of the upper cylinder body from the lower cylinder body. The chamber 20 of the lower cylinder body 16 is configured to slidably contain a needle array base holder 17.

Needle array base holder 17 is a solid cylinder with a solution supply lumen 13 and a suction lumen 6. The upper end of the holder 17 is fixedly attached to the upper cylinder body 12 with its suction and supply lumens aligned with the corresponding lumens in the upper cylinder body. A needle array base 7 is attached to a chamber in the bottom of the holder. Solution supply 13 and suction 6 lumens in the array base align with corresponding lumens 13 and 6 in the holder 17. A plurality of microneedles 10 extend downward from the array base 7.

Protective plate 11 attaches inside the lower end of the cavity 20 in the lower cylinder body 16 and forms the bottom of the tip assembly 25. Openings in the plate 11 are positioned to allow microneedles 10 to pass through, and larger openings 8 and 9 align with the suction 6 and solution supply 13 lumens in the array base 17.

FIG. 9 is a bottom view of applicator tip assembly 25. A plurality of ball bearings 23 are retained on protective plate 11 by means of ball bearing concavities 22 molded into the plate. The ball bearings 23 may be plastic, metal, glass or other material. A plurality of curved thermoplastic resin surface strips 24 protrude slightly from the protective plate 11.

In operation, the handpiece applicator tip interface 4 is inserted into the upper chamber 14 of the assembled applicator tip 15. Solution supply tube 18 and suction tube 19 align with solution lumen 13 and suction lumen 6 that run all the way through the apparatus to suction opening 8 and supply opening 9. The clinician activates the hydro dermabrasion machine and presses the applicator tip against the skin then pushes downward. Spring 21 is compressed by downward push of the hydro dermabrasion handpiece 3 toward the skin. Compression of spring 21 advances microneedle array base 7, which advances microneedles 10 past protective plate 11 and into the skin. When the clinician releases downward pressure, the spring decompresses, the needles retract, and the clinician shifts the tip to a new location. Using the trigger 2, the clinician may apply solution, suction, or both.

The ball bearings 23 reduce friction of the protective plate 11 against the skin while the needles are retracted. The surface strips 24 assist the liquid agent dispersion across the skin, and also help seal the cartridge against the skin to prevent suction loss and liquid leakage. If the clinician keeps the dual pump actuation trigger 2 depressed continuously, the suction function of the device will suction up the expressed solution and any skin cell remnants via suction aperture 8 in the assembly.

Once the clinician has completed the treatment, he/she simply detaches applicator tip assembly 15 from handpiece applicator tip interface 4 and disposes of the assembly.

In another embodiment, the applicator is modified to allow the operator to adjust the penetration/extension depth of the microneedles. In this embodiment, external, depth-calibrated, screw threads 36 are added to the outside diameter of the lower half of upper cylinder body 12. A depth selection indicator tic mark 37 is added to a second external ledge 41 around lower cylinder body 16. Internal screw groove threads 38 that correspond to external screw threads 36 are added to a new depth adjustment cylinder 39 which has a larger diameter than upper cylinder body 12, allowing it to be screwed onto upper cylinder body 12 in a clockwise direction. During assembly at the factory, depth adjustment cylinder 39 is screwed onto upper cylinder body 12 until the bottom 42 of depth adjustment cylinder 39 comes into contact with external ledge 41 of lower cylinder body 16. This is the “zero” position indicating that when the operator presses down, the needles will not extend past protective plate 11. At this time, the assembler affixes an adhesive depth measurement marking decal 40 around the lower half of depth adjustment cylinder 39 so that the “0.0 mm” mark on the decal aligns with depth selection indicator tic mark 37 on lower cylinder body 16. To increase the depth of penetration, the operator turns depth adjustment cylinder 39 counterclockwise until depth indicator tic mark 37 aligns with the desired depth measurement marking on depth measurement marking decal 40. Alternatives to a decal, such as engraving directly on the surface of depth adjustment cylinder 39, may be used to indicate selected penetration depth.

The foregoing description has been presented and is intended for the purposes of illustration and description. It is not intended to be exhaustive nor limit the invention to the above teachings. The embodiments were chosen and described in order to best explain the principles of the invention and its practical application and to enable others skilled in the art to best utilize the invention in various embodiments and with various modifications as are suited to the particular use contemplated. Numerous other changes, substitutions, variations, alterations, and modifications may be ascertained to one skilled in the art. Therefore, it is intended that the invention not be limited to the particular embodiments disclosed for carrying out the invention.

1. An applicator for use with a handpiece of a hydro dermabrasion device for treating skin, having solution supply and suction apertures, comprising:
a cylinder that connects to the handpiece, comprising an upper cylinder body with upper and lower walls and a lower cylinder body;
the upper cylinder body comprising an upper chamber, a lower chamber and a connecting chamber;
wherein the upper chamber has an open top and a bottom with a supply aperture and a suction aperture and is removably attachable to the handpiece, and the lower chamber and connecting chamber are connected and each has an open bottom;
The lower cylinder body being dimensioned to slidably fit inside the lower chamber of the upper cylinder body, and having a first ledge and a second ledge;
a microneedle array base holder, with a supply lumen and a suction lumen, attached to the bottom of the upper chamber of the upper cylinder body;
a microneedle array base attached to the holder, having supply and suction lumens therethrough and a plurality of microneedles extending therefrom;
a safety plate having apertures for the microneedles and supply and suction lumens therethrough, attached to the lower cylinder body below the microneedle array base;
a spring positioned between the lower wall of the upper cylinder body and the first ledge around the lower cylinder body; and
a microneedle penetration depth controller;
Wherein the supply apertures and supply lumens are aligned to form a continuous solution supply path through the applicator, the suction apertures and suction lumens form a continuous path through the applicator and the microneedles are reciprocally passable through the safety plate to the treated skin at a depth regulated by the depth controller.

2. The applicator of claim 1 wherein the safety plate includes a plurality of ball bearing concavities with ball bearings and a plurality of raised surface strips along a periphery between the ball bearings.

3. The applicator of claim 1 wherein the upper cylinder body includes external threads and the microneedle penetration depth controller comprises a depth adjustment cylinder fitting around the upper cylinder body, having a top and a bottom and internal threads that engage the external threads on the upper cylinder body.

4. The applicator of claim 3 wherein the depth controller includes a penetration depth indicator.

5. The applicator of claim 3 wherein the microneedles do not pass through the safety plate when the bottom of the depth adjustment cylinder is in contact with the second ledge around the lower cylinder body, and the depth of penetration of the microneedles increases as the depth adjustment cylinder is moved away from the second ledge.

6. The applicator of claim 4 wherein the depth adjustment cylinder is moved by threaded rotation.

7. The applicator of claim 6 wherein the safety plate includes a plurality of ball bearing concavities with ball bearings and a plurality of raised surface strips along a periphery between the ball bearings.
